(19) United States
(12) Patent Application Publication
(10) Pub. No.: US 2021/0162090 A1
(43) Pub. Date: Jun. 3, 2021

(54) COMPOSITE FIBER

(71) Applicant: National Central University, Taoyuan City (TW)

(72) Inventors: Wei-Wen Hu, Taipei City (TW); Yu-Ting Lin, Taichung City (TW)

(73) Assignee: National Central University, Taoyuan City (TW)

(21) Appl. No.: 16/699,138

(22) Filed: Nov. 29, 2019

(51) Int. Cl. A61L 15/22; A61K 33/38; A61K 38/18; A61L 15/44; D01D 5/00; D01F 9/04; D01F 6/62; D01F 1/10

(52) U.S. Cl. CPC A61L 15/225; D10B 2331/041; A61K 38/1858; A61L 15/44; D01D 5/003; D01F 9/04; D01F 6/625; D01F 1/103; A61L 2300/104; A61L 2300/252; A61L 2300/404; A61L 2300/414; D10B 2509/022; D10B 2401/13; A61K 33/38

(57) ABSTRACT

The present invention provides a composite fiber which comprises an alginate fiber, a polymer material, an antibacterial agent, and a plasmid encoding growth factor-gene. The present invention also provides a wound dressing, wherein the wound dressing comprises a composite fiber as described above. The composite fibers prepared according to the present invention are capable of releasing the antibacterial agent and the growth factor gene, not only to reduce microorganism growth, but also to secrete growth factors in a wound site through transfection, thereby promoting wound healing.

### FIELD OF THE INVENTION

The present invention discloses a composite fiber of an alginate fiber and a polymer material by using a co-electrospinning technique, which is characterized in that the composite fiber is loaded an antibacterial agent and a plasmid encoding growth factor-gene. The prepared fibers are capable of not only reducing microorganism growth, but also secreting growth factors in a wound site through in situ transfection to promote wound healing.

### BACKGROUND OF THE INVENTION

Traditional dressings are made of cotton or synthetic fibers, such as gauze, cotton sheets, etc., which have the advantages of quick absorption of wound exudates and simple processing, however, because the permeability is high, the wound is excessively dry, and microorganisms can easily pass through to cause infections, more importantly, they tend to stick to the wounds when they are removed, causing great pains to patients when the dressings are replaced.

The common form of synthetic dressings is a film made of polyurethane (PU), it is a type of waterproof dressings capable of keeping wounds moist and preventing bacteria from passing through, however, this type of dressings cannot absorb wound exudates and tends to cause damages to cells and tissues when they are peeled off.

These dressings only can provide passive protections, cannot promote wound regeneration. Accordingly, they cannot be used in chronic wounds which commonly occurred in diabetic patients.

In addition, dressings added with silver ions or nano-silver have also been used in medical devices to reduce the risk of infections. However, excessively high concentration of silver has been proved to be cytotoxic, which may retard wound healing and thus cannot be used to treat chronic wounds.

Most of the commercially available products mainly provide passive protection, though some of them emphasize antibacterial effects, they do not promote wound tissue regeneration.

In order to combine the advantages of the above-mentioned wound dressings and to overcome their shortcomings, there is a dire need of a novel multifunctional wound dressings.

Nanofibrous scaffolds can simulate the structure of extracellular matrices, in addition, it is believed that they can increase cell attachment, migration, differentiation, and proliferation. Further, since the nanofibrous scaffold has a big specific surface area and high porosity, it can provide a wound with better air permeability and protect the wound site against liquid accumulation, thereby having the ability to promote wound healing. Electrospinning technology is considered the simplest and the most cost effective method.

### DETAILED DESCRIPTION OF THE INVENTION

The composite fiber of the present invention is produced by methods such as electrospinning or electrospray.

Electrospun fibers used in the field of tissue engineering can be divided into two major categories: one made of natural polymers and one made of synthetic polymers, each of them has its own advantages and disadvantages.

Natural polymers are obtained from animals and plants, have good biocompatibility and biodegradability, but their poor mechanical properties limit the development of natural polymers; synthetic polymers are synthesized by polymerization of petroleum-based chemicals, have good mechanical properties, but their degraded byproducts may be cytotoxic.

Electrospun fibers have the characteristics of high specific surface area, small pore size, and high porosity, accordingly they have great potentials in many applications, wound dressings in particular.

It takes time for chronic wounds to be healed, which may cause many risks and inconveniences in daily life. Therefore, the present invention intends to develop a multifunctional wound dressing to promote tissue regeneration.

The composite fiber composition of the present invention comprises an alginate fiber, a polymer material, an antibacterial agent, and a plasmid encoding growth factor-gene.

In one embodiment, the antimicrobial agent is a metal ion, nanoparticle, or an oxide thereof, an antibiotic, graphene or carbon nanotubes or a combination thereof.

In one embodiment, the antimicrobial agent comprises silver ion, titanium dioxide nanoparticles, zinc oxide nanoparticles, copper oxide nanoparticles, iron tetroxide nanoparticles, nano-silver or a combination thereof.

In one embodiment, the polymer material is biodegradable.

In one embodiment, the polymer material comprises polyester, polyamide, polycarbonate, polyurethane, or a combination thereof.

In one embodiment, the polyester comprises polylactide (PLA), polyglycolic acid (PGA), polylactic-co-glycolic acid (PLGA), or polycaprolactone (PCL).

In one embodiment, the growth factor-gene is a gene encoding a platelet-derived growth factor, an epidermal growth factor, a keratinocyte growth factor, a fibroblast growth factor, a transforming growth factor-131, a vascular endothelial growth factor, an insulin-like growth factor, or a combination thereof.

In one embodiment, a hydrophilic alginate fiber has high absorbance, is capable of absorbing wound exudates and providing a moist environment, and the polymer material is capable of increasing mechanical strength and promoting cell adhesion.

The present invention introduces an antibacterial agent into the polymer material so as to continuously inhibit microorganism growth.

In one embodiment, the plasmid encoding the growth factor-gene of the present invention is encapsulated by a non-viral vector.

In another embodiment, the non-viral vector comprises a liposome complex, a cationic polymer, a peptide, or a chitosan polymer.

In another embodiment, non-viral vector and the plasmid form a positively charged complex.

In one embodiment, the non-viral vector of the present invention is adsorbed onto the alginate fiber, wherein the non-viral vector encapsulates the plasmid encoding growth factor-gene.

In another embodiment, the positively charged complex of the present invention adheres onto the alginate fibers by the electrostatic interaction.

In one embodiment, the weight ratio of the alginate fiber and the polymer material of the present invention may range from 1:9 to 9:1.

In one embodiment, the weight ratio of the alginate fiber and the polymer material is 8:2.

In one embodiment, a calcium salt is used by the present invention to crosslink the alginate fiber.

In one embodiment, the calcium salt comprises calcium carbonate, calcium phosphate, calcium oxalate, calcium chloride, calcium sulfate or calcium nitrate.

The alginate used in the present invention is easily soluble in water, and it is used to cross-linking with calcium ions so that an egg box structure is formed to avoid alginate fibers dissolve in water, and the crosslinked calcium ions can be released to promote blood clotting.

The present invention can be used as dressings for wound healing, first of all, the composite fiber has the properties of high specific surface area and high porosity to provide a wound with high air permeability, the composition of components comprises an antibacterial agent and a plasmid encoding growth factor-gene, and calcium ions which can achieve multifunctions of bacteria inhibition, wound repair, and blood coagulation, respectively.

The present invention further provides a method for producing a composite fiber, wherein the method step comprises:

step (a) providing an alginate solution and a polymer material solution, wherein alginate and polyoxyethylene (PEO) or polyvinyl alcohol (PVA) are mixed to obtain a solution having a concentration of 1 to 10 wt % of alginate, preferably an alginate solution of 2 to 8 wt %; polymer material and polyoxyethylene (PEO) or polyvinyl alcohol (PVA) are mixed to obtain a polymer material solution; step (b) providing a nano-silver solution, wherein the nano-silver solution is formed through a redox reaction of a silver salt and a reducing agent; step (c) mixing the nano-silver solution with the polymer material solution to obtain a silver-loaded polymer solution; and step (d) producing the composite fiber from the alginate solution and the silver-loaded polymer solution.

In one embodiment, the step (d) comprises an electro spinning technique or an electrospray technique.

In one embodiment, step (d) further comprises solution-loaded syringes arranged in two auto-sampling devices at a sampling rate of 0.1 to 5.0 mL/h, wherein via a voltage of 12 to 24 kV, and at a collection distance of 10 to 25 cm to collect nanofibers through a co-electrospinning technology.

In one embodiment, the method for producing a composite fiber may further comprise a step (e) of adsorbing positively charged complexes formed by combining a non-viral vector and a plasmid onto the composite fiber produced in step (d).

In one embodiment, wherein the silver salt refers to a generic term of ionic compounds formed of anionic ions and silver ions, comprising silver acetate, silver nitrite, silver nitrate, silver chloride, or silver sulfate.

In one embodiment, wherein the reducing agent comprises sodium borohydride, hydrazine hydrate, sodium citrate, or dimethylformamide.

In one embodiment, the nano-silver solution is prepared by a mechanical ball milling method, an evaporation-condensation method, a photochemical reduction method, a liquid chemical reduction method, an electrochemical reduction method, a liquid redox method, a microemulsion method or a chemical precipitation method.

In one embodiment, wherein the concentration of the nano-silver solution is from 5 mM to 75 mM.

In one embodiment, wherein the concentration of the nano-silver solution is 30 mM.

### BRIEF DESCRIPTION OF THE DRAWINGS

FIG. 1 is a schematic drawing showing the preparation of a composite fiber by a co-electrospinning technology.

FIG. 2 is an SEM image showing the appearance of the composite fibers.

FIG. 3 is a TEM image showing the nano-silver in a composite fiber.

FIG. 4 is a photograph showing the antibacterial effect of the composite fibers at different proportions against Staphylococcus epidermidis by using a disk diffusion method.

FIG. 5 is a photograph showing the antibacterial effect of the composite fibers at different proportions against Escherichia coli by using a paper ingot diffusion method.

FIG. 6 is a graph showing the analysis of the bactericidal rates of the composite fibers at different proportions.

FIG. 7 is a graph showing the analysis of the bactericidal rates of the composite fibers having different concentrations of nano-silver.

FIG. 8 is a graph showing the analysis of the survival rates of NIH 3T3 cells cultured in the composite fibers at different proportions.

FIG. 9 is a graph showing the analysis of the survival rates of NIH 3T3 cells cultured in the composite fibers having different concentrations of nano-silver after 1 and 5 days.

FIG. 10 is a fluorescence photograph showing in situ transfection of cells in the composite fibers at a ratio of A8P2.

FIG. 11 is a fluorescence photograph showing in situ transfection of cells in the composite fibers at a ratio of A2P8.

FIG. 12 is a graph comparing the coagulation rates of the composite fibers.

FIG. 13 is a graph comparing the appearances of wounds treated with different composite fibers on days 7 and 11.

FIG. 14 is a graph comparing the wound healing rates treated with different composite fibers on day 7 and day 11.

FIG. 15 showing H&E staining images of sections of wound tissues treated with different composite fibers on day 7 and day 11.

FIG. 16 is a schematic diagram showing the multifunctions of the composite fiber.

### EXAMPLES

Production of Composite Fibers

Preparation of Alginate/Polyethylene Oxide (PEO) Spinning Solution

A 5 g of spinning solution was prepared by mixing 3.33 g of alginate stock solution, 1.0 g of PEO stock solution and 0.525 g of co-solvent (dimethyl sulfoxide)/surfactant (Triton X-100), and adding 0.145 g of water, so that the final concentration of alginate was 4 wt %, PEO was 2 wt %, dimethyl sulfoxide was 10%, and Triton X-100 was 0.5%, and the solution was heated and stirred (at 50° C., 60 rpm) for 2 days, the bubbles were removed by centrifugation.

Preparation of PCL/PEO Solution

4 g of solution was prepared from 1.8 g of polycaprolactone (PCL) stock solution and 1.8 g of PEO stock solution, and then 0.4 g of dimethylformamide (DMF) was added to obtain a solution, of which the final concentration of PCL was 4.5 wt %, PEO was 3.6 wt %, then the solution was heated and stirred (40° C., 60 rpm) for one day.

Preparation of 30 mM of Ag PCL/PEO Spinning Solution

25.48 mg of silver nitrate was added to 0.5 ml of dimethylformamide (DMF) and stirred at 60 rpm for 5 min at room temperature, then 0.4 ml of silver-containing DMF solution was added dropwise to 3.6 g of PCL/PEO solution, the solution was finally stirred and heated at 40° C., 60 rpm for one day to complete the preparation.

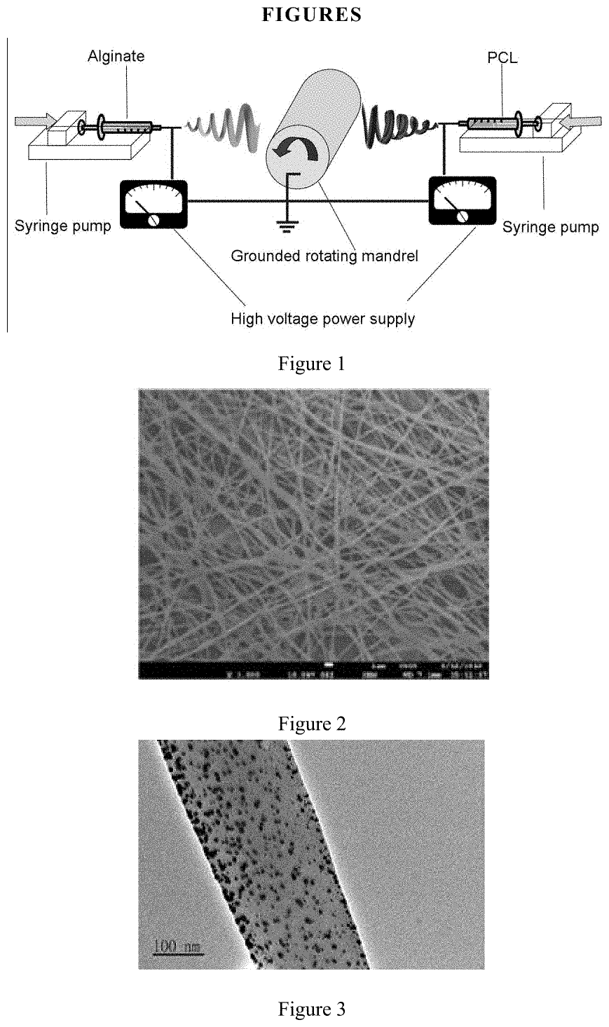

The composite fiber of alginate spinning solution and Ag PCL/PEO spinning solution was synthesized by co-electrospinning (FIG. 1).

In the present invention, nano-silver was introduced into PCL, and then co-electrospun with alginate to form the composite fibers (FIG. 2). It was confirmed that the composite fibers were indeed a nano-network structure and the PCL had nano-silver (FIG. 3).

In order to increase the effect of wound healing, the platelet-derived growth factor B (PDGF B) was added to the composite fibers in the present invention, wherein PDGF B was a chemoattractant of neutrophils and capable of inducing the proliferation and differentiation of fibroblasts, which in turn promoted wound repair.

In the manufacturing method, plasmid DNA encoding PDGF B-gene was encapsulated by cationic polymer to form positively charged complex, which was adsorbed onto the negatively-charged alginate fiber in the composite fiber.

Antibacterial Experiments

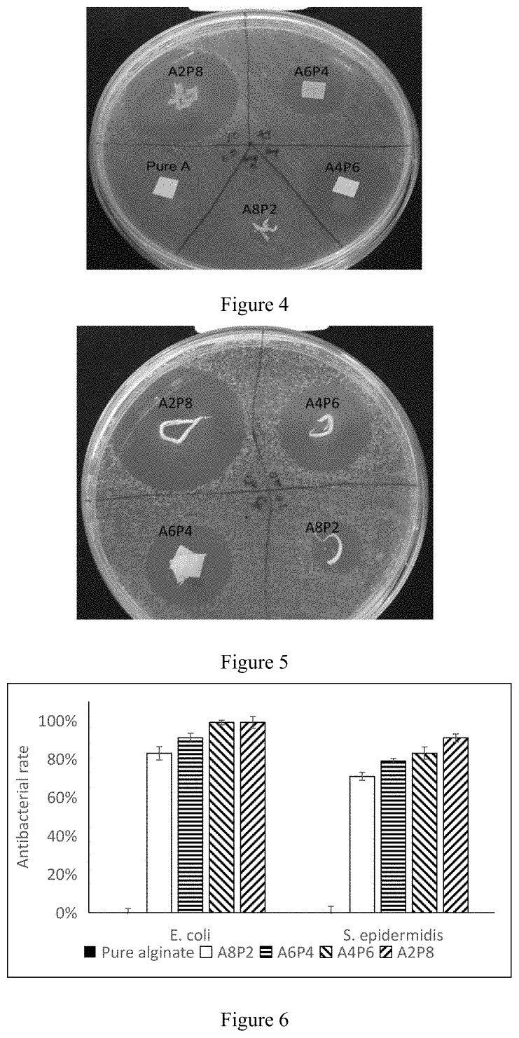

In the present invention, nano-silver was introduced into the PCL fibers, and the composite fibers produced from alginate/PCL at a weight ratio of 8:2 (A8P2), 6:4 (A6P4), 4:6 (A4P6), and 2:8 (A2P8) were subjected to antibacterial experiments. It was found that the growth of Staphylococcus epidermidis (FIG. 4) and Escherichia coli (FIG. 5) were inhibited, and the effect increased with an increase in the proportion of PCL, and the inhibitory effect was effectively achieved even with only 20% of PCL (A8P2), whereas pure alginate (pure A) fiber showed no antibacterial effect.

In the present invention, the composite fibers produced from alginate/PCL at a weight ratio of 8:2 (A8P2), 6:4 (A6P4), 4:6 (A4P6), and 2:8 (A2P8) were subjected to tests for bactericidal rate of Staphylococcus epidermidis and Escherichia coli, and comparisons to pure alginate were also conducted. Even with only 20% of PCL (A8P2), a bactericidal rate of 83% of Staphylococcus epidermidis and 71% of Escherichia coli were able to be achieved after 12 hours (FIG. 6).

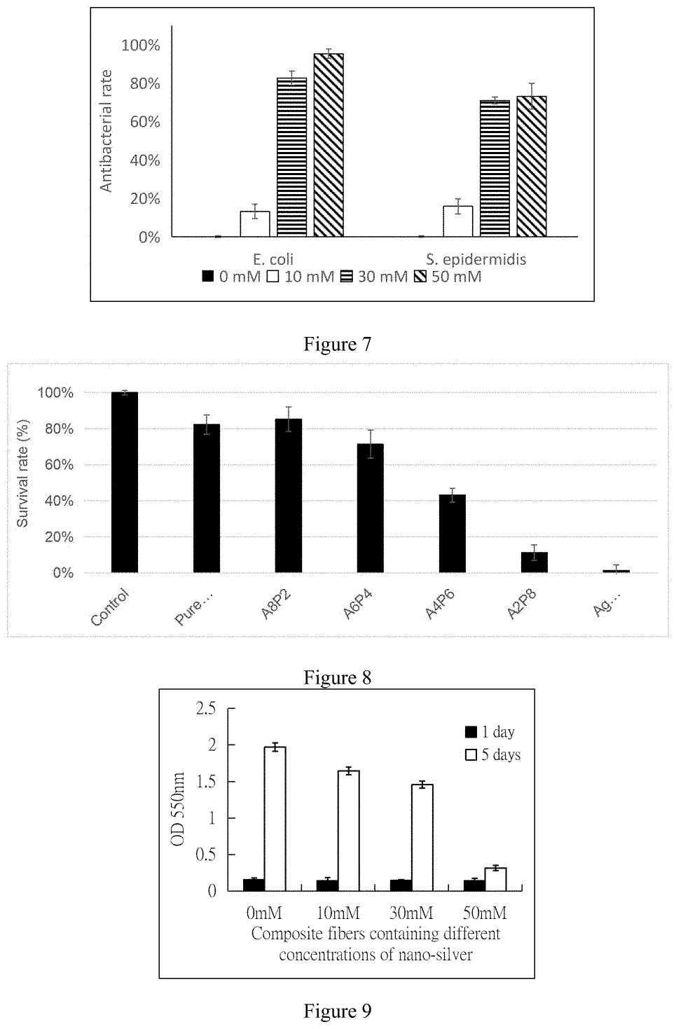

In the present invention, the composite fibers having a concentration of 0 mM, 10 mM, 30 mM, and 50 mM of nano-sliver were subjected to evaluate their bactericidal rate against Escherichia coli and Staphylococcus epidermidis. After 11.5 hours, the Escherichia coli bactericidal rates of the composite fibers having 30 mM and 50 mM of nanosilver were 83% and 95%, respectively, and the Staphylococcus epidermidis bactericidal rate were 71% and 73%, respectively (FIG. 7), both of which were over 70%.

Cell Survival Rate Test (MTT Assay)

Since the release of nano-silver from composite fibers might cause cytotoxicity, the cell survival rate test was performed using the MTT assay. It was found that the higher the proportion of PCL, the more significant the toxicity of nano-silver, however, A6P4 and A8P2 were able to maintain more than 60% of cell survival rates (FIG. 8).

NIH 3T3 cells were cultured on the composite fibers for 1 and 5 days, and the cell survival rate was analyzed by MTT. Compared to the control group on day 5 (FIG. 9), it was found that the cytotoxicity of composite fibers containing 50 mM of nano-silver was very significant, but no significant difference was found for composite fibers containing 10 mM and 30 mM of nano-silver.

Although composite fibers containing 50 mM of nano-silver had the best antibacterial effect, the cytotoxicity of this concentration was too high to be suitable for wound dressing.

On the other hand, plasmid DNA containing genes of green fluorescent protein and PDGF B was encapsulated by positively charged non-viral vector to form a positively charged complex, and adsorbed onto the composite fibers for in situ transfection.

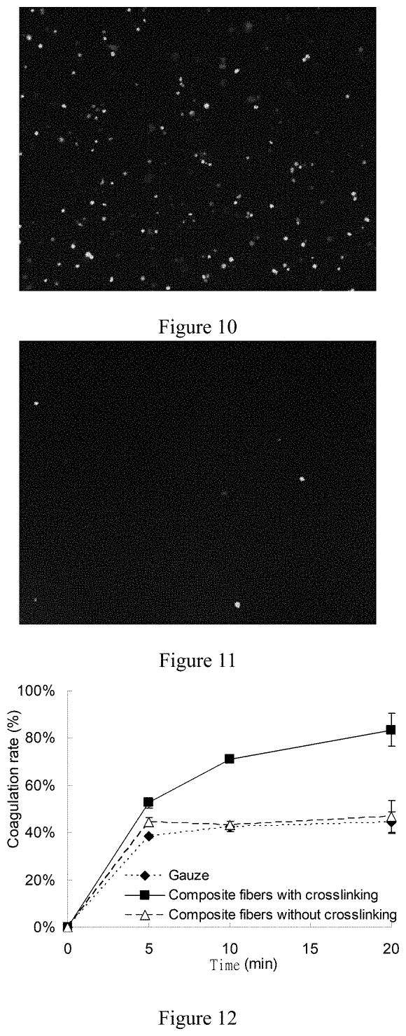

Since alginate was negatively charged, it was able to promote the adsorption of positively charged complexes. The results showed that the higher the proportion of alginate, the better the transfection effect (FIGS. 10 and 11).

The results confirmed that the present invention was able to regulate the composition ratio of the fiber, thereby controlling the composite fiber to have both antibacterial and gene delivery capabilities, avoiding the side effect of cytotoxicity caused by the antibacterial nano-silver. The ratio of A8P2 was the one that had the best overall performance in this embodiment.

Blood Coagulation Test

Since slow coagulation might hinder wound healing and increase the risk of infection, blood coagulation function of the composite fiber was tested. 100 μl of human whole blood containing anticoagulants was first added to the composite fibers, placed at room temperature for 5, 10, and 20 minutes, and then the blood coagulation rate was measured spectrometrically (FIG. 12).

Because the crosslinked composite fiber was able to release calcium ions, the coagulation rate was significantly higher than that of gauze and uncrosslinked composite fibers.

Wound Healing Test

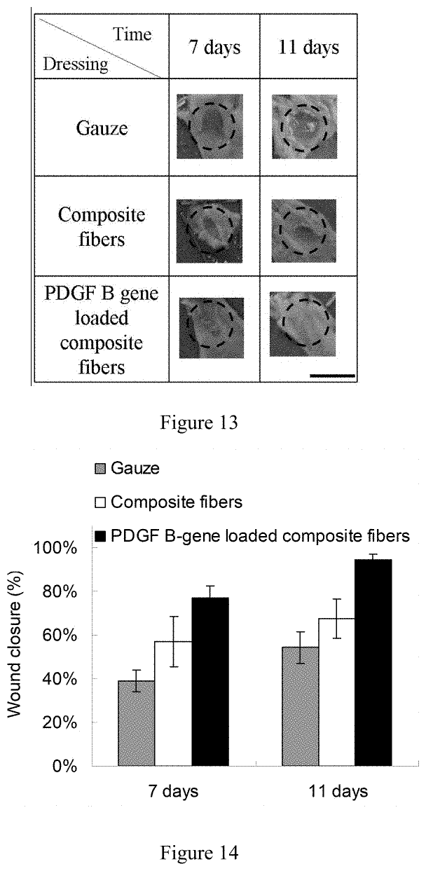

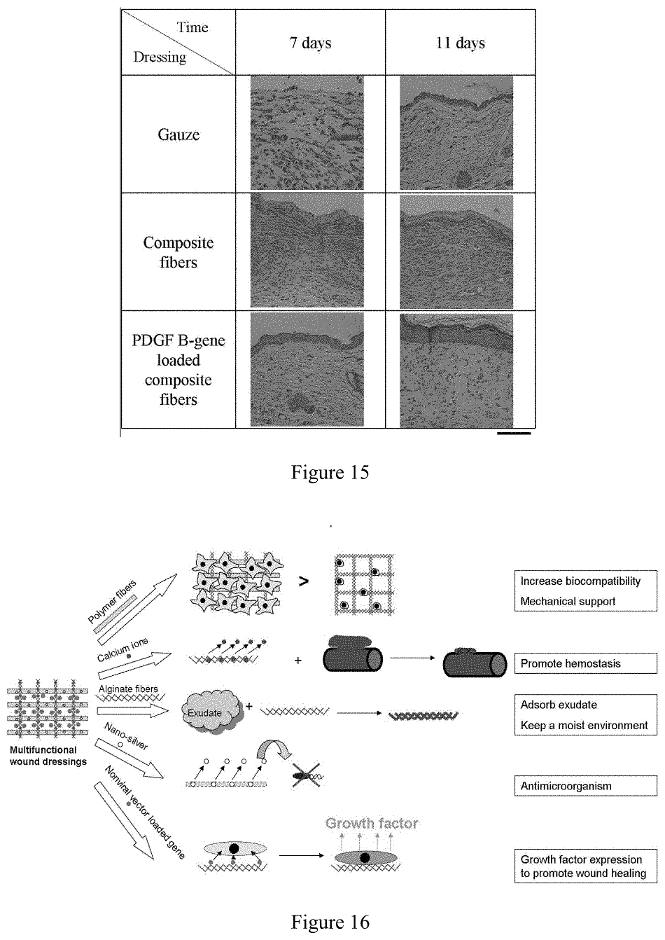

Two 5 mm-diameter wounds were created on the back of C57BL/6 mice, the wound dressings were placed on the wounds, the wound sizes were recorded on day 7 and day 11, respectively. Based on the wound appearance (FIG. 13) and wound healing rate (FIG. 14), it was found that the wound healing of the control group (wounds without dressing coverage), even after 11 days, was less than 60%. In contrast, the PDGF gene-loaded composite fibers caused wound healing of 77% and 95% on day 7 and day 11, respectively, and the wound was almost invisible after 11 days. After being stained by H & E staining (FIG. 15)), it was found that the epidermis had formed in the PDGF gene-loaded composite fiber group on day 7, suggesting that PDGF B gene was able to deliver to the wound site, so that the transfected cells secreted PDGF B to promote wound healing.

In sum, the present invention, after the above-described tests, showed that the composite fiber had high mechanical strength, and the functions of hemostasis acceleration, wound exudate absorption, bacteria inhibition and promotion of wound tissue regeneration (FIG. 16), and the composition ratio of the components was adjustable to make the composite fiber to perform even better.

Although the present invention has been disclosed using the above-mentioned preferred embodiments, it is not intended to limit the present invention. It will be readily apparent to a person skilled in the art that varying substitutions and modifications may be made to the invention disclosed herein without departing from the scope and spirit of the invention. Therefore, the scope of protection of the present invention shall be determined by the scope of the appended claims.

What is claimed is:

1. A composite fiber, wherein the composite fiber comprises an alginate fiber, a polymer material, an antibacterial agent and at least one plasmid encoding growth factor-gene.

2. The composite fiber of claim 1, wherein the antimicrobial agent is a metal ion, nanoparticle, or an oxide thereof, an antibiotic, graphene or carbon nanotubes or a combination thereof.

3. The composite fiber of claim 1, wherein the polymer material comprises polyester, polyamide, polycarbonate, polyurethane, or a combination thereof.

4. The composite fiber of claim 1, wherein the growth factor-gene is a gene encodes a platelet-derived growth factor, an epidermal growth factor, a keratinocyte growth factor, a fibroblast growth factor, a transforming growth factor-β1, a vascular endothelial growth factor, an insulin-like growth factor growth factor or a combination thereof.

5. The composite fiber of claim 1, wherein the weight ratio of the alginate fiber and the polymer material ranges from 1:9 to 9:1.

6. The composite fiber of claim 5, wherein the weight ratio of the alginate fiber and the polymer material is 8:2.

7. The composite fiber of claim 2, wherein the antibacterial agent is nano-silver.

8. The composite fiber of claim 1, wherein the alginate fiber crosslink by using a calcium salt.

9. The composite fiber of claim 8, wherein the calcium salt is calcium carbonate, calcium phosphate, calcium oxalate, calcium chloride, calcium sulfate or calcium nitrate.

10. The composite fiber of claim 1, wherein the plasmid encoding growth factor-gene is encapsulated by a non-viral vector.

11. The composite fiber of claim 10, wherein the non-viral vector comprises a liposome complex, a cationic polymer, a peptide or a chitosan polymer.

12. A method for producing a composite fiber, wherein the method step comprises:
step (a) providing an alginate solution and a polymer material solution, wherein alginate and polyoxyethylene (PEO) or polyvinyl alcohol (PVA) are mixed to obtain a solution having a concentration of 1 to 10 wt % of alginate, preferably a alginate solution of 2 to 8 wt %; polymer material and polyoxyethylene (PEO) or polyvinyl alcohol (PVA) are mixed to obtain a polymer material solution;
step (b) providing a nano-silver solution, wherein the nano-silver solution is formed through a redox reaction of a silver salt and a reducing agent;
step (c) mixing the nano-silver solution with wherein the polymer material solution to obtain a silver-loaded polymer solution; and
step (d) producing the composite fiber from the alginate solution and the silver-loaded polymer solution.

13. The method for producing a composite fiber of claim 12, which further comprises a step (e) of adsorbing positively charged complexes formed by combining a non-viral vector and a plasmid onto the composite fiber produced in step (d).

14. The method for producing a composite fiber of claim 12, wherein the concentration of the nano-silver solution ranges from 5 mM to 75 mM.

15. The method for producing a composite fiber of claim 14, wherein the concentration of the nano-silver solution is 30 mM.

16. The method for producing a composite fiber of claim 12, wherein the reducing agent comprises sodium borohydride, hydrazine hydrate, sodium citrate or dimethylformamide.
